(19) United States
(12) Patent Application Publication
(10) Pub. No.: US 2010/0121253 A1
(43) Pub. Date: May 13, 2010

(54) Method and Apparatus of Low Strength Electric Field Network-Mediated Delivery of Drug, Gene, SI-RNA, SH-RNA Protein, Peptide, Antibody or Other Biomedical and Therapeutic Molecules and Reagents in Solid Organs

(75) Inventor: Luyi Sen, Stevenson Ranch, CA (US)

Correspondence Address: Law Offices of Daniel L. Dawes;Dawes Patent Law Group, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/531,294

(22) PCT Filed: Mar. 12, 2008
(86) PCT No.: PCT/US08/56693

§ 371 Date: Sep. 17, 2009

Related U.S. Application Data
(60) Provisional application No. 60/894,877, filed on Mar. 14, 2007.

(51) Int. Cl. A61N 1/30; A61K 31/7088; A61K 38/16; A61K 38/00; A61K 39/395

(52) U.S. Cl. 604/20; 514/44 A; 514/44 R; 514/12; 514/2; 424/130.1; 604/501

(57) ABSTRACT

A methodology and apparatus for drug, gene, siRNA, shRNA shRNA peptide, protein, antibody or any other biomedical therapeutic reagents targeting in various of solid organs of large animals and humans ex vivo and in vivo is described. The delivery is assisted with the application of a low strength electric field network (LSEN). A low strength electric field network (LSEN) system is used for ex vivo or in vivo delivery of any therapeutic gene, siRNA, shRNA shRNA protein or drugs into a lung, pleura, breast, liver, spleen, pancreas, kidney, adrenal, prostate, testicle, ovary and tumors in chest, abdominal and pelvic cavity.

The present application is related to U.S. Provisional Patent Application Ser. No. 60/894,877, filed on Mar. 14, 2007, which is incorporated herein by reference and to which priority is claimed pursuant to 35 USC 119.

### BACKGROUND OF THE INVENTION

1. Field of the Invention

The invention relates to apparatus and methods using low strength electroporation networks (LSEN).

2. Description of the Prior Art

Electroporation is a method involving the application of short duration, high intensity electric field pulses to cells or tissue. The electrical stimuli cause membrane destabilization and the subsequent formation of nanometer-sized pores in the cellular membrane. In this permeabilized state, the membrane can allow passage of DNA, enzymes, antibodies and other macromolecules into the cell. On the other hand, electric pulses with high electric field intensity can cause permanent cell membrane breakdown or cell lysis. Therefore, only less than 10 pulses were typically used for in vitro or in vivo gene or drug delivery. For more than two decades, the use of this technique had been restricted to suspension of cultured cells only, since the electric pulses are administered in a pair of electrode in a small cuvette.

Most recently, various types of plate-needle electrodes have been developed, and electro-injection of chemical or foreign genes was applied in animal tissues in vivo, such as skin, skeletal muscle, and tumors. For whole organ in vivo gene transfer, a few attempts were made in liver of rats, mice and cats using a direct method of gene injection using single- or six-needle inserted electrodes. Plate-needle type electrodes were even been used in a whole chicken embryo and in a rodent's vein graft ex vivo electroporation to facilitate reporter gene transfer.

There are only two reports regarding myocardium electroporation using cuvette type electrodes. In hearts of early chicken embryos, reporter gene delivered ex vivo yielded the strongest intensity of gene expression compared to two other non-viral gene transfer methods, microparticle bombardment and lipofection. Wang et al. immersed the mice heart into buffer with DNA/dendrimer and applied electroporation in a cuvette. The efficiency of reporter gene transfer was increased 200 fold.

Electroporation holds great potential not only in gene therapy, but also in other areas such as transdermal drug delivery and chemotherapy. However, safety is a major concern. All of the previous studies used high voltage for electroporation. The strength of the electrical field is usually between 250-1000 V/cm. A recent study shows so called “low voltage” electroporation, 100 V/cm, can cause 100 fold rise in IL-5 production in mice skeletal muscle after IL-5 gene injection. Most recently, a conventional pair of needles was used to apply 200 v/cm electroporation pulses after plasmid mIL-10 direct injection into the rat's bilateral tibialis anterior muscles. Serum mIL-10 level was increased 100 fold on day 2 and this level was doubled on day 6 which significantly attenuated myocardial lesions and improved hemodynamic parameters in an experimental autoimmune myocarditis rat model. However, more than 10 kV is needed to electropermeabilize the large animal or human heart.

The electroporation apparatus used for gene delivery used two needles or electrode plates to apply high voltage, short duration pulses on the mice tumor model. This system caused significant tissue damage and inflammation due to the needle direct injury and the high voltage shock that limited its use. A microchip device published recently for skin electroporation that will also use high voltage although it has not been used in human animal yet.

Although, the efficiency is high, a new device, methodology and optimum conditions of electro-gene transfer needs to be established for the application of electropermeabilization in a whole organ of large animal and human, such as heart and liver and the like.

### BRIEF SUMMARY OF THE INVENTION

The illustrated embodiment of the invention comprises a methodology and apparatus for drug, gene, siRNA, shRNA, peptide, protein, antibody or any other biomedical therapeutic reagents targeting several solid organs of large animals and humans ex vivo and in vivo assisted with the application of a low strength electric field network (LSEN). LSEN meshes and electropermeabilization methodologies are disclosed in Provisional Patent Application Ser. No. 60/744,522, filed: Apr. 10, 2006 and Provisional Patent Application Ser. No. 60/819,277, filed: Jul. 6, 2006, both of which are incorporated herein by reference (hereinafter called LSEN applications). LSEN is properly referred to as a low strength electropermeabilizing field network rather than low strength electroporating field network, because at the low voltage levels which LSEN uses the biomechanism is believed to be qualitatively different than in conventional high voltage electroporation. It is currently understood that LSEN may not generate as many or as large a pore in the cell membrane as it increases cell membrane activity and permeability.

It is to be understood, however, that the LSEN meshes and electrodes and their combinations are structurally altered according to the present invention to be adapted for optimum use for each of the solid organs and tissues disclosed and claimed in the present application. For example, the LSEN meshes and electrodes and their combinations for use with the liver are specially arranged and configured for creating an LSEN field in the liver depending on whether the application is ex vivo, in vivo and where the latter, whether it is used inside or outside the body. Similarly, the shape and size of the LSEN meshes and electrodes and their combinations for use with the lung or portions thereof will be structurally altered to be optimal for that application as opposed to the shape and sized used with the liver. Further, it is to be understood that there is considerable individual variation in organ size and shape from one patient to another. Therefore, individualization of shape and size is to be expected, certainly between infant, juvenile and adult patients as well as having a design and construction which is customizable at the site of application by the surgeon. For example, a negative mesh of a universal size and shape can be constructed so that it is capable of being trimmed to size and shape for each individual application.

This invention includes, but is not limited to, the following embodiments, namely a method and apparatus for gene, protein and drug delivery into the lung, pleura, breast, liver, spleen, pancreas, kidney, adrenal tissue, prostate, testicles, ovaries and/or tumors. The gene, protein and drug delivery preferably occurs during and after application of LSEN, but the scope of the invention contemplates that delivery also is performed before application of LSEN. In any case, transfer of the gene, protein and drug into the cells of tissue mass occurs in relation to LSEN application of the cells.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is an anatomical depiction of the practice of the illustrated embodiment of the invention in the lung in vivo.

FIG. 2 is an anatomical depiction of the practice of the illustrated embodiment of the invention in a lobe of the lung ex vivo.

FIG. 3 is an anatomical depiction of the practice of the illustrated embodiment of the invention in the pleura.

FIGS. 4.I and 4.IIa-4.IIc are anatomical depictions of the practice of the illustrated embodiment of the invention in the breast.

FIGS. 5.Ia, 5.Ib, 5.IIa and 5.IIb are anatomical depictions of the practice of the illustrated embodiment of the invention in the liver in vivo.

FIGS. 6.Ia, 6.Ib, 6.IIa, 6.IIb and 6.III are anatomical depictions of the practice of the illustrated embodiment of the invention in the liver ex vivo.

FIGS. 7.I, 7.IIa, and 7.IIb are anatomical depictions of the practice of the illustrated embodiment of the invention in the kidney.

FIGS. 8, 8.Ia, and 8.Ib are anatomical depictions of the in vivo practice of the illustrated embodiment of the invention in the pancreas.

FIGS. 9.I, 9.II, and 9.III are anatomical depictions of the practice of the illustrated embodiment of the invention in the ex vivo LSEN of the pancreas.

FIGS. 10.Ia, 10.Ib, 10.Ic, 10.IIa, 10.IIb and 10.III are anatomical depictions of the in vivo practice of the illustrated embodiment of the invention in the venous system of the kidney.

FIGS. 11.I, 11.II, and 11.III are anatomical depictions of the ex vivo practice of the illustrated embodiment of the invention in the venous system of the kidney.

FIGS. 12.I, 12.IIa, 12.IIb and 12.III are anatomical depictions of the practice of the illustrated embodiment of the invention in adrenal tissue.

FIGS. 13.Ia, 13.Ib, 13.IIa, 13.IIb, 13.III, 13.IIIa, and 13.IIIb are anatomical depictions of the practice of the illustrated embodiment of the invention in the prostate.

FIGS. 14.Ia, 14.Ib, 14.IIa, and 14.III are anatomical depictions of the practice of the illustrated embodiment of the invention in the testicle.

FIGS. 15.Ia, 15.Ib, 15.IIa, and 15.III are anatomical depictions of the practice of the illustrated embodiment of the invention in the ovary.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment is a method and apparatus for low strength electropermeabilization LSEN-mediated gene, protein and drug delivery in the isolated organs and tissue ex vivo, vessels and tissue in vivo. As a proof of concept, we conducted a series studies using LSEN system of the illustrated embodiment for gene delivery in large animal hearts ex vivo and in vivo. We found the method of the illustrated embodiment has the highest gene transfer efficiency and efficacy. It is higher than any existing viral and nonviral gene transfer techniques. We did not find any cardiac and adverse effect in large animal to date.

The illustrated embodiment of the invention introduces a new strategy for electro-permeabilization the cell membrane for gene, protein, drug targeting in skin, soft tissue and bone ex vivo and in vivo, that is to use an array of electrodes to apply the electric field network with low voltage, short pulse duration, burst pulses for a long period time. The illustrated embodiment has been demonstrated as a method of using low voltage pulses on 11 different types of solid organs or tissue masses, each realizing the same transfection efficiency.

The list of molecules and their inhibitors, enhancers, regulator, genes, siRNAs, shRNAs, antigens, antibodies, and peptides that are related with these molecules, that can be used in the invention for the arthritis and other orthopedic diseases is extensive. The use of some of these agents in immune suppression in combination with LSEN is disclosed in copending application Ser. No. ______, filed on ______ entitled, “A Method For Using Low Strength Electric Field Network (LSEN) And Immunosuppressive Strategies To Mediate Immune Responses”, which is incorporated herein by reference. The following list is to be understood as illustrative and not limiting with respect to the possible transfected materials using the invention.
1) Cytokines:
   a) Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.
   b) Other Cytokines: AREG, BMP1, BMP2, BMP3, BMP7, CAST, CD40LG, GERI, CKLFSF1, CKLFSF2, CLC, CSF1, CSF2, CSF3, CTF1, CXCL16, EBI3, ECGF1, EDA, EPO, ERBB2, ERBB21P, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, 1L1B, 1L1F10, IL1F5, IL1F6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGBIA1, SCGB3A1, SCYE1, SDCBP, SECTMI, SIVA, SLCOIA2, SLURP1, SOCS2, SPP1, SPREDI, SRGAP1, THPO, TNF, TNFRSFI1B, TNFSF10, TNFSFI1, TNFSF13, TNFSFI3B, TNFSF14, TNFSFI5, TNFSF18, TNFSF4, TNFSF7, TNFSF8, TNFSF9, TRAP1, VEGF, VEGFB, YARS.
2) Cytokine Receptors:
   a) Cytokine Receptors: CNTFR, CSF2RA, CSF2RB, CSF3R, EBI3, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, 1L22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.
   b) Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.
3) Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.
4) Cytokine Production: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7.
5) Other Genes involved in Cytokine-Cytokine Receptor Interaction: ACVR1, ACVR1B, ACVR2, ACVR2B, AMH, AMHR2, BMPR1A, BMPR1B, BMPR2, CCR1, CD40, CRLF2, CSFIR, CXCR3, IL18RAP, IL23R, LEP, TGFB1, TGFB2, TGFB3, TGFBR1, TGFBR2, TNFRSFIA, TNFRSF1B, TNFRSF21, TNFRSF8, TNFRSF9, XCR1.
6) Acute-Phase Response: AHSG, APCS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3.
7) Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, IL22, IL31RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3C1, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.
8) Humoral Immune Response: BATF, BCL2, BF, BLNK, C1R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSFIR, CSF2RB, CXCR3, CYBB, EBI3, FADD, GPI, IL10, IL12A, IL12B, IL12RB1, IL13, IL18, IL1B, 1L2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.
9) Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC
10) Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin)
11) Cell adhesion molecule: ICAM1, ITGA1, ITGA2, ITGA3, ITGAM, ITGAV, ITGB1, VCAM1
12) Skeletal Development:
   a) Bone Mineralization: AHSG, AMBN, AMELY, BGLAP, ENAM, MGP, MINPP1, SPP1, STATH, TUFT1.
   b) Cartilage Condensation: BMP1, COL11A1, MGP, SOX9.
   c) Ossification: ALPL, AMBN, AMELY, BGLAP, CALCR, CASR, CDH11, DMP1, DSPP, ENAM, IBSP, MGP, MINPP1, PHEX, RUNX2, SOST, SPARC, SPP1, STATH, TFIP11, TUFT1.
   d) Osteoclast Differentiation: BGLAP, TWIST2.
   e) Other Genes Involved in Skeletal Development: ARSE, BMP2, BMP3, BMP4, BMP5, BMP6, BMP7, BMP8B, COL10A1, COL12A1, COL1A1, COL1A2, COL2A1, COL9A2, COMP, FGFR1, FGFR3, GDF10, IGF1, IGF2, MSX1, MSX2, TWIST1.
13) Bone Mineral Metabolism:
   a) Calcium Ion Binding and Homeostasis: ANXA5, ARSE, BGLAP, BMP1, CALCR, CASR, CDH11, COMP, DMP1, EGF, MGP, MMP13, MMP2, MMP8, SPARC, VDR.
   b) Phosphate Transport: COL10A1, COL11A1, COL12A1, COL14A1, COL15A1, COL16A1, COL17A1, COL18A1, COL19A1, COL1A1, COL1A2, COL2A1, COL3A1, COL4A3, COL4A4, COL4A5, COL5A1, COL7A1, COL9A2.
14) Cell Growth and Differentiation:
   a) Regulation of the Cell Cycle: EGFR, FGF1, FGF2, FGF3, IGF1R, IGF2, PDGFA, TGFB1, TGFB2, TGFB3, VEGF, VEGFB, VEGFC.
   b) Cell Proliferation: COL18A1, COL4A3, CSF3, EGF, EGFR, FGF1, FGF2, FGF3, FLT1, IGF1, IGF1R, IGF2, PDGFA, SMAD3, SPP1, TGFB1, TGFB2, TGFB3, TGFBR2, VEGF, VEGFB, VEGFC.
   c) Growth Factors and Receptors: BMP1, BMP2, BMP3, BMP4, BMP5, BMP6, BMP7, BMP8B, BMPR1A, CSF2, CSF3, EGF, EGFR, FGF1, FGF2, FGF3, FGFR1, FGFR2, FGFR3, FLT1, GDF10, IGF1, IGF1R, IGF2, PDGFA, SPP1, TGFB1, TGFB2, TGFB3, TGFBR1, TGFBR2, VEGF, VEGFB, VEGFC.
   d) Cell Differentiation: SPP1, TFIP11, TWIST1, TWIST2.
15) Extracellular Matrix (ECM) Molecules:
   a) Basement Membrane Constituents: COL4A3, COL4A4, COL4A5, COL7A1, SPARC.
   b) Collagens: COL10A1, COL11A1, COL12A1, COL14A1, COL15A1, COL16A1, COL18A1, COL19A1, COL1A1, COL1A2, COL2A1, COL3A1, COL4A3, COL4A4, COL4A5, COL5A1, COL7A1, COL9A2.
   c) ECM Protease Inhibitors: AHSG, COL4A3, COL7A1, SERPINH1.
   d) ECM Proteases: BMP1, CTSK, MMP10, MMP13, MMP2, MMP8, MMP9, PHEX.
   e) Structural Constituents of Bone: BGLAP, COL1A1, COL1A2, MGP.
   f) Structural Constituents of Tooth Enamel: AMBN, AMELY, ENAM, STATH, TUFT1.
   g) Other ECM Molecules: BGN, BMP2, BMP8B, COL17A1, COMP, CSF2, CSF3, DCN, DSPP, EGF, FGF1, FGF2, FGF3, FLT1, GDF10, IBSP, IGF1, IGF2, PDGFA, SPP1, VEGF, VEGFB.
16) Cell Adhesion Molecules:
   a) Cell-cell Adhesion: CDH11, COL11A1, COL14A1, COL19A1, ICAM1, ITGB1, VCAM1.
   b) Cell-matrix Adhesion: ITGA1, ITGA2, ITGA3, ITGAM, ITGAV, ITGB1, SPP1.
   c) Other Cell Adhesion Molecules: BGLAP, CD36, COL12A1, COL15A1, COL16A1, COL18A1, COL4A3, COL5A1, COL7A1, COMP, FN1, IBSP, SCARB1, TNF.
17) Transcription Factors and Regulators: MSX1, MSX2, NFKB1, RUNX2, SMAD1, SMAD2, SMAD3, SMAD4, SMAD5, SMAD6, SMAD7, SMAD9, SOX9, TNF, TWIST1, TWIST2, VDR.
18) Skeletal Development:
   a) Bone Mineralization: AHSG, AMBN, AMELY, BGLAP, ENAM, MINPP1, STATH, TUFT1.
   b) Cartilage Condensation: BMP1, COL11A1, SOX9.
   c) Ossification: ALPL, AMBN, AMELY, BGLAP, CALCR, CDH11, DMP1, DSPP, ENAM, MINPP1, PHEX, RUNX2, STATH, TFIP11, TUFT1.
   d) Osteoclast Differentiation: BGLAP.
   e) Other Genes Involved in Skeletal Development: BMP2, BMP3, BMP4, BMP5, BMP6, COL10A1, COL12A1, COL1A1, COL1A2, COL2A1, COMP, FGFR1, GDF10, IGF1, IGF2, MSX1, TWIST1.
19) Bone Mineral Metabolism:
   a) Calcium Ion Binding and Homeostasis: ANXA5, BGLAP, BMP1, CALCR, CDH11, COMP, DMP1, EGF, MMP2, MMP8, VDR.
   b) Phosphate Transport: COL10A1, COL11A1, COL12A1, COL14A1, COL15A1, COL1A1, COL1A2, COL2A1, COL3A1, COL4A3, COL5A1.
20) Cell Growth and Differentiation:
   a) Regulation of the Cell Cycle: EGFR, FGF1, FGF2, FGF3, IGF1R, IGF2, PDGFA, TGFB1, TGFB2, TGFB3, VEGF, VEGFB.
   b) Cell Proliferation: COL4A3, CSF3, EGF, EGFR, FGF1, FGF2, FGF3, FLT1, IGF1, IGF1R, IGF2, PDGFA, SMAD3, TGFB1, TGFB2, TGFB3, TGFBR2, VEGF, VEGFB.
   c) Growth Factors and Receptors: BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGF, VEGFB.
   d) Cell Differentiation: TFIP11, TWIST1.
21) Extracellular Matrix (ECM) Molecules:
   a) Basement Membrane Constituents: COL4A3.
   b) Collagens: COL10A1, COL11A1, COL12A1, COL14A1, COL15A1, COL1A1, COL1A2, COL2A1, COL3A1, COL4A3, COL5A1.
   c) ECM Protease Inhibitors: AHSG, COL4A3, SERPINH1.
   d) ECM Proteases: BMP1, CTSK, MMP10, MMP2, MMP8, MMP9, PHEX.
   e) Structural Constituents of Bone: BGLAP, COL1A1, COL1A2.
   f) Structural Constituents of Tooth Enamel: AMBN, AMELY, ENAM, STATH, TUFT1.
   g) Other ECM Molecules: BGN, BMP2, COMP, CSF2, CSF3, DSPP, EGF, FGF1, FGF2, FGF3, FLT1, GDF10, IGF1, IGF2, PDGFA, VEGF, VEGFB.
22) Cell Adhesion Molecules:
   a) Cell-cell Adhesion: CDH11, COL11A1, COL14A1, ICAM1, ITGB1, VCAM1.
   b) Cell-matrix Adhesion: ITGA1, ITGA2, ITGA3, ITGAM, ITGB1.
   c) Other Cell Adhesion Molecules: BGLAP, CD36, COL12A1, COL15A1, COL4A3, COL5A1, COMP, FN1, SCARB1, TNF.
23) Transcription Factors and Regulators: MSX1, NFKB1, RUNX2, SMAD1, SMAD2, SMAD3, SMAD4, SOX9, TNF, TWIST1, VDR.
24) Skeletal Development:
   a) Bone Mineralization: AHSG, AMBN, AMELY, BGLAP, ENAM, MGP, MINPP1, SPP1, STATH, TUFT1.
   b) Cartilage Condensation: BMP1, COL11A1, MGP, SOX9.
   c) Ossification: ALPL, AMBN, AMELY, BGLAP, CALCR, CASR, CDH11, DMP1, DSPP, ENAM, IBSP, MGP, MINPP1, PHEX, RUNX2, SOST, SPARC, SPP1, STATH, TFIP11, TUFT1.
   d) Osteoclast Differentiation: BGLAP, TWIST2.
   e) Other Genes Involved in Skeletal Development: ARSE, BMP2, BMP3, BMP4, BMP5, BMP6, BMP7, BMP8B, COL10A1, COL12A1, COL1A1, COL1A2, COL2A1, COL9A2, COMP, FGFR1, FGFR3, GDF10, IGF1, IGF2, MSX1, MSX2, TWIST1.
25) Bone Mineral Metabolism:
   a) Calcium Ion Binding and Homeostasis: ANXA5, ARSE, BGLAP, BMP1, CALCR, CASR, CDH11, COMP, DMP1, EGF, MGP, MMP13, MMP2, MMP8, SPARC, VDR.
   b) Phosphate Transport: COL10A1, COL11A1, COL12A1, COL14A1, COL15A1, COL16A1, COL17A1, COL18A1, COL19A1, COL1A1, COL1A2, COL2A1, COL3A1, COL4A3, COL4A4, COL4A5, COL5A1, COL7A1, COL9A2.
26) Cell Growth and Differentiation:
   a) Regulation of the Cell Cycle: EGFR, FGF1, FGF2, FGF3, IGF1R, IGF2, PDGFA, TGFB1, TGFB2, TGFB3, VEGF, VEGFB, VEGFC.
   b) Cell Proliferation: COL18A1, COL4A3, CSF3, EGF, EGFR, FGF1, FGF2, FGF3, FLT1, IGF1, IGF1R, IGF2, PDGFA, SMAD3, SPP1, TGFB1, TGFB2, TGFB3, TGFBR2, VEGF, VEGFB, VEGFC.
   c) Growth Factors and Receptors: BMP1, BMP2, BMP3, BMP4, BMP5, BMP6, BMP7, BMP8B, BMPR1A, CSF2, CSF3, EGF, EGFR, FGF1, FGF2, FGF3, FGFR1, FGFR2, FGFR3, FLT1, GDF10, IGF1, IGF1R, IGF2, PDGFA, SPP1, TGFB1, TGFB2, TGFB3, TGFBR1, TGFBR2, VEGF, VEGFB, VEGFC.
   d) Cell Differentiation: SPP1, TFIP11, TWIST1, TWIST2.
27) Extracellular Matrix (ECM) Molecules:
   a) Basement Membrane Constituents: COL4A3, COL4A4, COL4A5, COL7A1, SPARC.
   b) Collagens: COL10A1, COL11A1, COL12A1, COL14A1, COL15A1, COL16A1, COL18A1, COL19A1, COL1A1, COL1A2, COL2A1, COL3A1, COL4A3, COL4A4, COL4A5, COL5A1, COL7A1, COL9A2.
   c) ECM Protease Inhibitors: AHSG, COL4A3, COL7A1, SERPINH1.
   d) ECM Proteases: BMP1, CTSK, MMP10, MMP13, MMP2, MMP8, MMP9, PHEX.
   e) Structural Constituents of Bone: BGLAP, COL1A1, COL1A2, MGP.
   f) Structural Constituents of Tooth Enamel: AMBN, AMELY, ENAM, STATH, TUFT1.
   g) Other ECM Molecules: BGN, BMP2, BMP8B, COL17A1, COMP, CSF2, CSF3, DCN, DSPP, EGF, FGF1, FGF2, FGF3, FLT1, GDF10, IBSP, IGF1, IGF2, PDGFA, SPP1, VEGF, VEGFB.
28) Cell Adhesion Molecules:
   a) Cell-cell Adhesion: CDH11, COL11A1, COL14A1, COL19A1, ICAM1, ITGB1, VCAM1.
   b) Cell-matrix Adhesion: ITGA1, ITGA2, ITGA3, ITGAM, ITGAV, ITGB1, SPP1.
   c) Other Cell Adhesion Molecules: BGLAP, CD36, COL12A1, COL15A1, COL16A1, COL18A1, COL4A3, COL5A1, COL7A1, COMP, FN1, IBSP, SCARB1, TNF.
29) Transcription Factors and Regulators: MSX1, MSX2, NFKB1, RUNX2, SMAD1, SMAD2, SMAD3, SMAD4, SMAD5, SMAD6, SMAD7, SMAD9, SOX9, TNF, TWIST1, TWIST2, VDR.

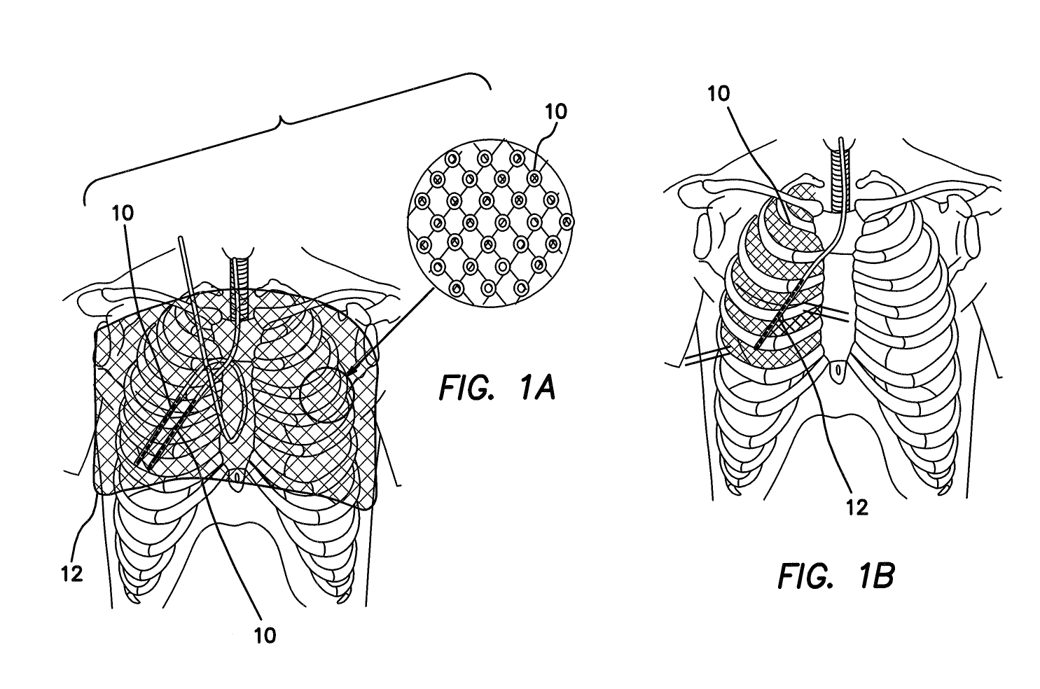

Turn first to a method for ex vivo or in vivo drug, gene, siRNA, shRNA, peptide, protein, antibody or any other biomedical therapeutic reagents in the lung as illustrated in FIG. 1. In this LSEN system, a positive electrode or electrodes 10 are designed in such a form and configuration to allow them to be placed into the vessels in the lung, for example the pulmonary artery or one or more of its branches by percutaneously insertion or directly placed in to pulmonary artery or its one or more of its branches during surgery. A negative electrode array 12 is designed in such a form and configuration to allow them to be placed on the out side of the lung through thoracoscopy or directly placed on the lung during open-chest surgery as illustrated in the left portion of FIG. 1. The term “electrodes” and an “electrode array” or “electrode mesh” shall be used interchangeably in this specification and shall refer to a collection or set of spatially arranged electrodes, such as, but not limited to, those which are disclosed in the incorporated LSEN applications above. Drug, gene, siRNA, shRNA, peptide, protein, antibody or any other biomedical therapeutic reagents is infused into the targeted lung through a pulmonary artery catheter 14 as shown in FIG. 1, which may also carry the positive electrode array 10. During the drug infusion, a low strength electric field network will be applied for in vivo gene, protein and drug delivery in the lung.

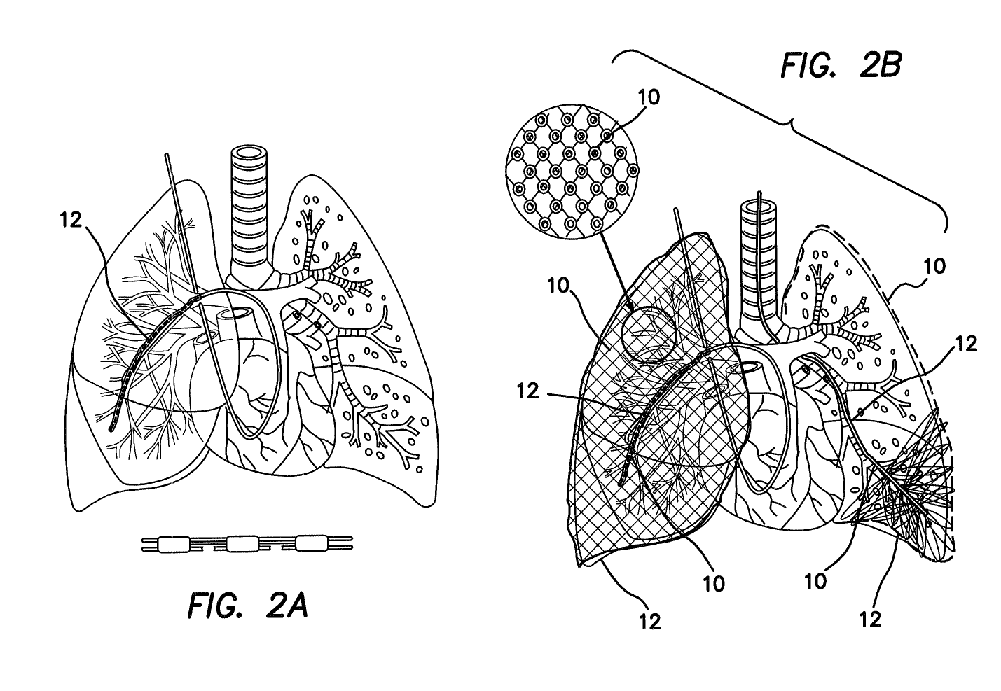

This method and apparatus can be modified as shown in FIG. 2 for localized in vivo gene, protein and drug delivery for one or two lobes, a part of the lobe, one lung, a part of the lung or two lungs by changing the size of the electrode arrays 10, 12, and the position of the positive electrode(s) 10 and the choice of the vessel for gene, protein and drug delivery.

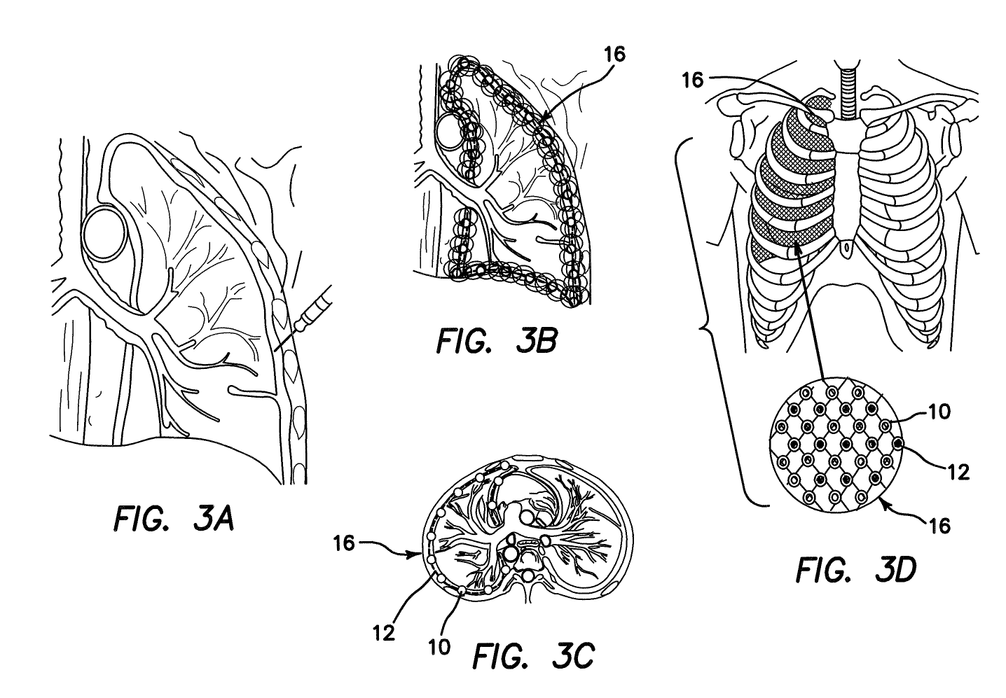

Alternatively, the positive electrode(s) 10 are placed into the respiratory tract, such as trachea, bronchus, bronchiole or alveolar duct instead of pulmonary artery or vein as shown in the right side of FIGS. 1 and 2. Similarly, the in vivo gene, protein and drug can also be delivered through respiratory tract instead of vessels. Another way is inject gene, protein or drug directly into the pleura cavity as shown in FIG. 3.

Alternatively, the negative electrode array 12 can also be noninvasively placed on the outside of the chest as shown in the right portion of FIG. 3, instead of into the chest cavity. The positive electrode(s) 10 are placed into the pulmonary vessels or respiratory tracts.

The positive and negative electrode positions are exchanged in each of the above scenarios according the physical and chemical characteristic of drug, gene, siRNA, shRNA, peptide, protein, antibody or any other biomedical therapeutic molecules and reagents for obtaining the optimal delivery efficiency.

Consider now ex vivo drug, gene, siRNA, shRNA, peptide, protein, antibody or any other biomedical therapeutic molecules and reagents in the lung. For ex vivo drug, gene, siRNA, shRNA, peptide, protein, antibody or any other biomedical therapeutic molecules and reagents in the lung, such as in lung transplantation, the negative electrode array 12 are directly placed on the outside surface of the lung as shown in the right side of FIG. 2. The positive electrode(s) 10 are placed either into the vessels of the lung, or the respiratory tract of the lung. The drug, gene, siRNA, shRNA, peptide, protein, antibody or any other biomedical therapeutic molecules and reagent are infused into either the vessels of the lung, or the respiratory tract of the lung as in the in vivo example.

Similarly, the positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

As stated above in one embodiment delivery of the drug, gene, siRNA, shRNA, peptide, protein, antibody or any other biomedical therapeutic molecules and reagents in the pleura is contemplated in FIG. 3. In this embodiment, the negative electrode array 12 can also be noninvasively placed on the outside of the chest, instead of into the chest cavity. The positive electrode(s) 10 are placed into the pulmonary vessels or respiratory tracts. The drug, gene, siRNA, shRNA, peptide, protein, antibody or any other biomedical therapeutic molecules and reagents are directly injected into the pleura cavity as shown in the left side of FIG. 3. Thus, LSEN is applied on the pleura during and after drug, gene, siRNA, shRNA, peptide, protein, antibody or any other biomedical therapeutic molecules and reagents injection. The electric field fringe travels across the chest wall, exterior and parietal pleura and peripheral parts of the lung. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

Alternatively, a positive and negative electrodes 10, 12 are alternatively spatially arranged so that the electrode array mesh 16 is designed to be placed into the chest cavity thoraciscopically as shown in FIG. 3. During and after drug, gene, siRNA, shRNA, peptide, protein, antibody or any other biomedical therapeutic molecules and reagents are directly injected into the pleura cavity, and the electric field is applied on the pleura. The electric field fringe extends parallel with the pleura and only across the pleural, not into the chest wall and less lung tissue is exposed or involved.

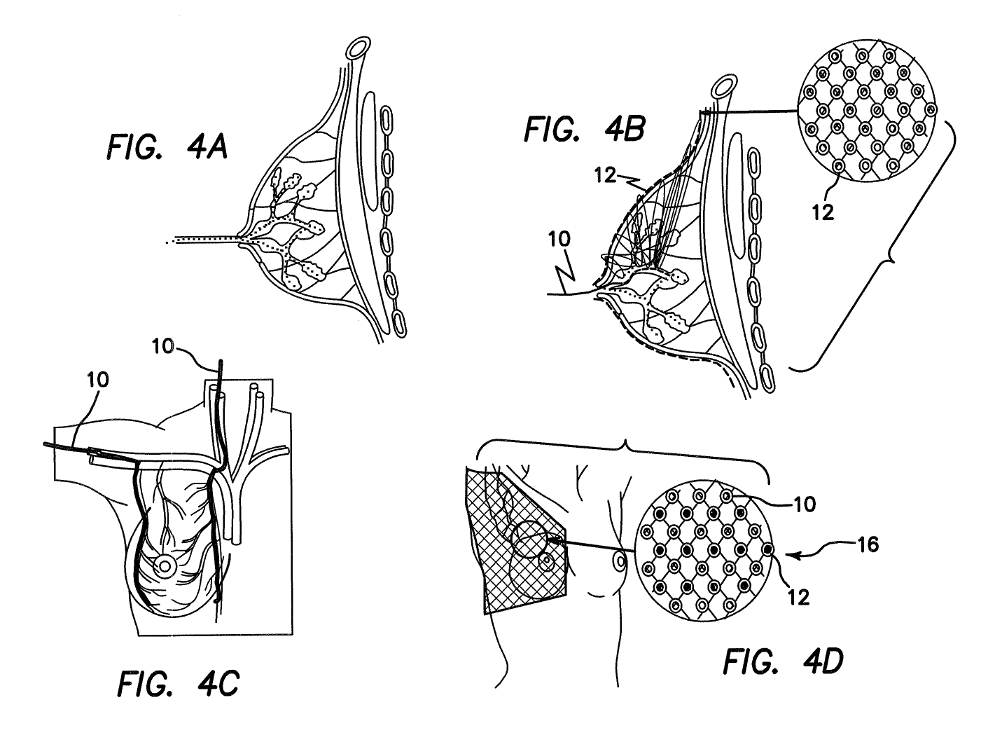

Consider now the method for drug, gene, siRNA, shRNA, peptide, protein, antibody or any other biomedical therapeutic molecules and reagents delivery in the breast as shown in FIGS. 4.I and 4.IIa-4.IIc. In this embodiment, the negative electrode array 12 is placed on the whole or partial breast in contact with skin as shown in FIG. 4.IIIa. The positive electrode(s) 10 are placed into the proximate vessels as shown in FIG. 4.IIb. Still further an alternating array 16 of positive and negative electrodes 10, 12 are placed in contact with the skin surface of the breast as shown in FIG. 4.IIc. The drug, gene, siRNA, shRNA, peptide, protein, antibody or any other biomedical therapeutic molecules and reagents are infused into the vessels or directly injected into the targeted area of breast. During the infusion or after the injection, LSEN is applied on the whole or part of the breast which was targeted.

Alternatively, drug, gene, siRNA, shRNA, peptide, protein, antibody or any other biomedical therapeutic molecules and reagents are infused into the milk duct as shown in FIG. 4.Ia. The positive electrode(s) 10 can also be placed into the milk ducts as shown in FIG. 4.IIa. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

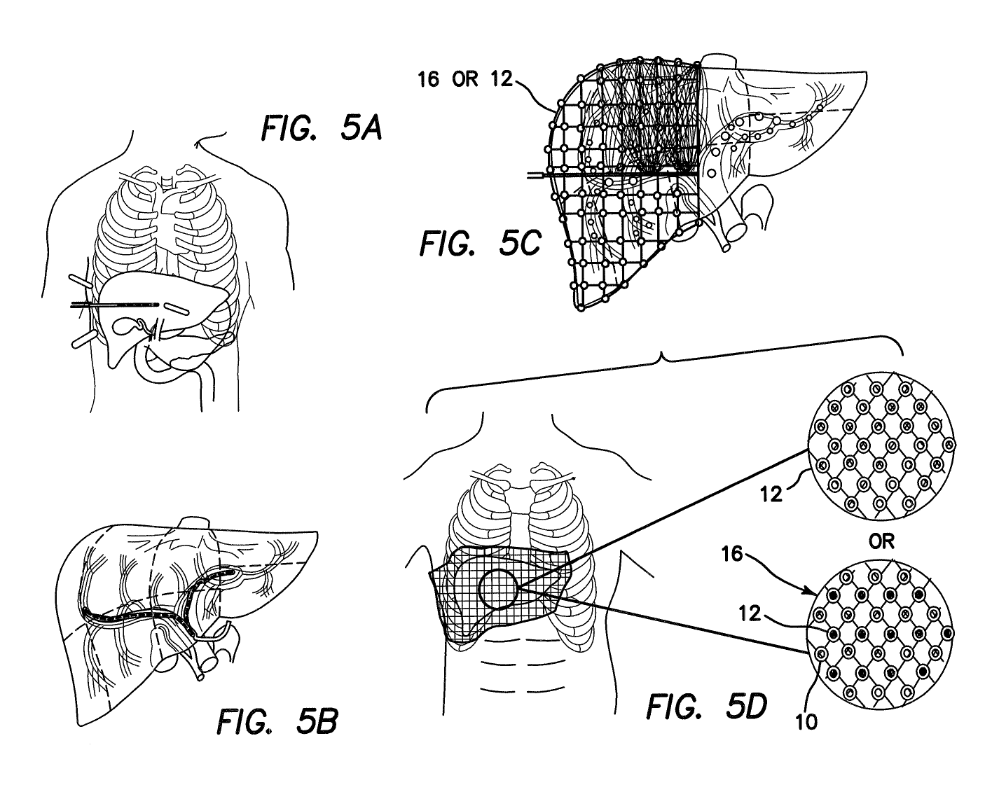

Similarly, consider a method for drug, gene, siRNA, shRNA, peptide, protein, antibody or any other biomedical therapeutic molecules and reagents delivery in the liver as shown in FIGS. 5.Ia, 5.Ib, 5.IIa and 5.IIb. In this embodiment, the negative electrode array 12 are placed on the surface of the liver endoscopically or during the abdominal surgery as shown in FIG. 5.IIa. The positive electrode(s) 10 are placed in vessels of the liver, such as hepatic artery(s) or vein(s) percutaneously as shown in FIG. 5.Ia or 5.Ib. The drug, gene, siRNA, shRNA, peptide, protein, antibody or any other biomedical therapeutic molecules and reagents are infused into the hepatic vessels. During and after the drug infusion, the electric field is applied on the targeted whole or part of the liver. The electric field network extends through the liver tissue. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

Alternatively, the negative electrode array mesh 12 is placed on the skin or body surface in the area of the liver as shown in FIG. 5.IIb. Thus, the electric field also includes part of the abdominal wall, but the procedure is less invasive.

Another alternative is to put the positive electrode(s) 10 into the portal vein as shown in FIG. 5.Ia. The drug, gene, siRNA, shRNA, peptide, protein, antibody or any other biomedical therapeutic molecules and reagents are infused into portal vein.

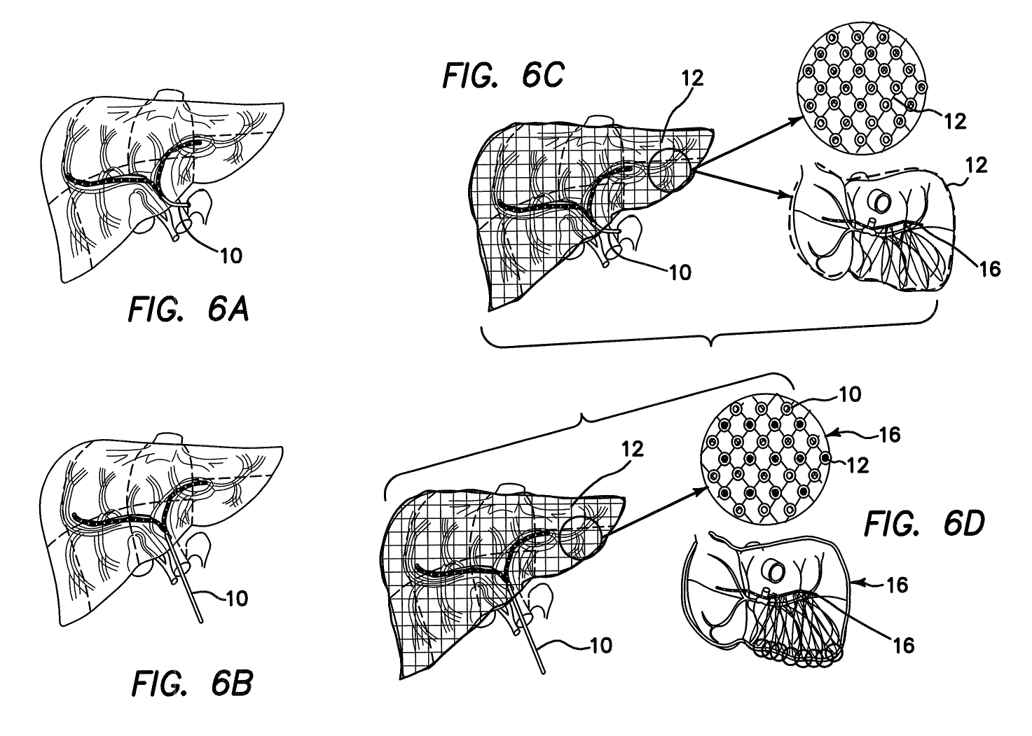

Turn to ex vivo drug, gene, siRNA, shRNA, peptide, protein, antibody or any other biomedical therapeutic molecules and reagents in the liver as shown in FIGS. 6.Ia, 6.Ib, 6.IIa, 6.IIb and 6.III. When the liver is outside of the body, the negative electrode array 12 is placed on the surface of the liver directly as shown in FIGS. 6.IIa and 6.IIb. The positive electrode(s) 10 are placed in vessels of the liver, such as hepatic artery(s), vein(s) or portal vein as shown in FIGS. 6.Ia, 6.Ib, 6.IIa and 6.IIb. The drug, gene, siRNA, shRNA, peptide, protein, antibody or any other biomedical therapeutic molecules and reagents are infused into the hepatic vessels. During and after the drug infusion, the electric field is applied on the whole or targeted part of the liver. The electric field network extends across the liver tissue.

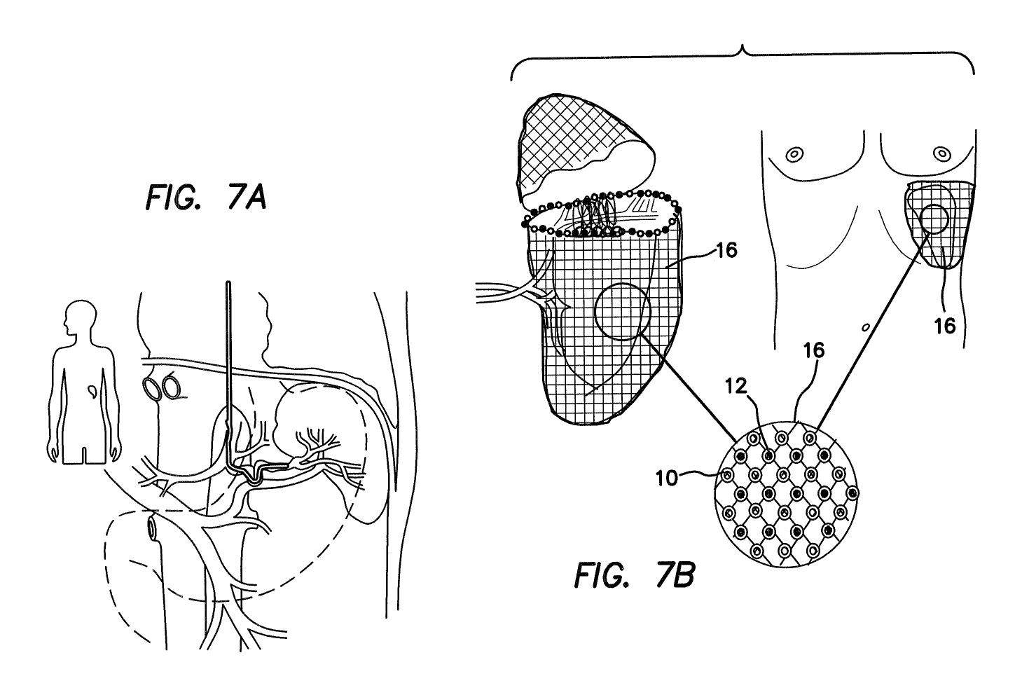

Similarly the method is used for drug, gene, siRNA, shRNA, peptide, protein, antibody or any other biomedical therapeutic molecules and reagents delivery in spleen as shown in FIGS. 7.I, 7.IIa, and 7.IIb. In one embodiment, the negative electrode array 12 is placed on the surface of the spleen endoscopically or during the abdominal surgery. The positive electrode(s) 10 are placed in splenic vessels percutaneously or directly. The drug, gene, siRNA, shRNA, peptide, protein, antibody or any other biomedical therapeutic molecules and reagents are infused into the splenic vessels. During and after the drug infusion, the electric field is applied to the whole or targeted part of the spleen. The electric field network extends through the spleen tissue. FIGS. 7.IIa, and 7.IIb illustrate an embodiment in which are bipolar array 16 is employed ex vivo or in vivo on the spleen or in contact with the skin in the proximity of the spleen. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

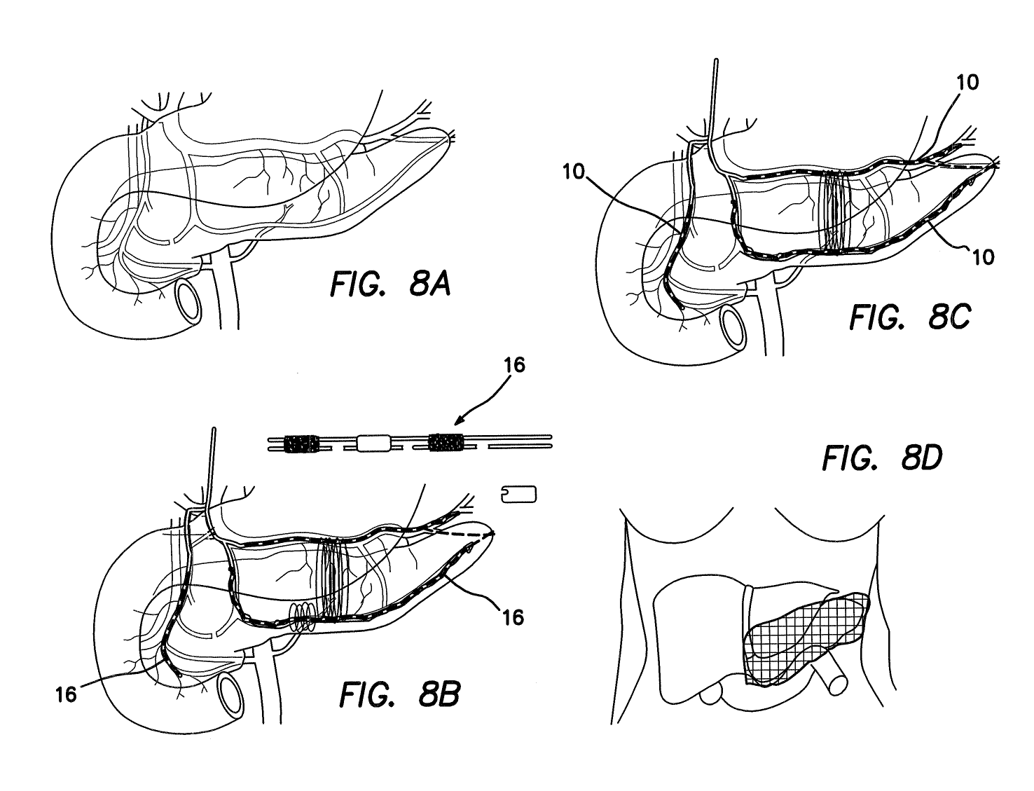

Again consider a method for drug, gene, siRNA, shRNA, peptide, protein, antibody or any other biomedical therapeutic molecules and reagents delivery in pancreas FIGS. 8, 8.Ia, and 8.Ib. In this embodiment, the negative electrode array 12 is placed on the surface of the pancreas endoscopically or during the abdominal surgery or in FIG. 8.Ib on the body surface in the proximate area. The positive electrode(s) 10 are placed in vessels of the pancreas percutaneously. In the embodiment of FIG. 8.Ia a catheter or catheters with a alternating linear array 16 of positive and negative electrodes is employed. The drug, gene, siRNA, shRNA, peptide, protein, antibody or any other biomedical therapeutic molecules and reagents are infused into the pancreatic vessels. During and after the drug infusion, the electric field is applied to the whole targeted pancreas. The electric field network extends through the pancreas tissue. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

Alternatively, the positive electrode(s) 10 are placed through a pancreatic duct during abdominal surgery or through the intestines. The drug, gene, siRNA, shRNA, peptide, protein, antibody or any other biomedical therapeutic molecules and reagents are also infused into the pancreatic duct.

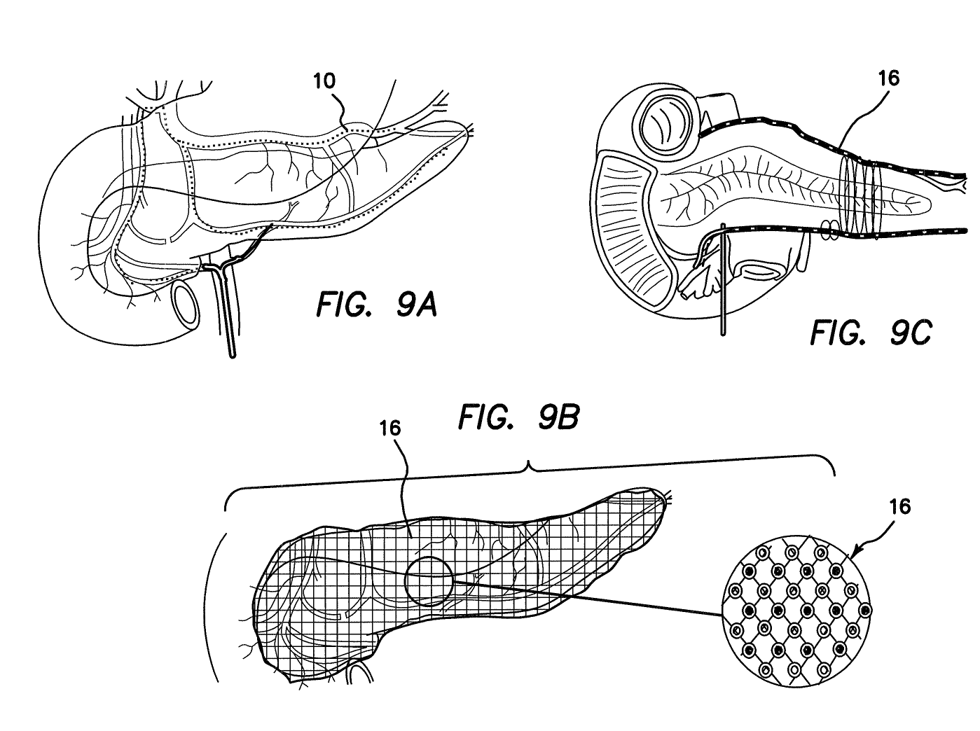

For an ex vivo application in FIGS. 9.I, 9.II, and 9.III, the negative electrode array 12 is placed on the surface of the pancreas directly. The positive electrode(s) 10 are placed in pancreatic vessels or duct. Alternatively, an alternating array 16 of positive and negative electrodes are applied to the surface of the pancreas as shown in FIG. 9.II. The drug, gene, siRNA, shRNA, peptide, protein, antibody or any other biomedical therapeutic molecules and reagents are infused into the pancreatic vessels or duct. During and after the drug infusion, the electric field is applied on the targeted whole or part of the pancreas. The electric field network extends across the pancreas tissue.

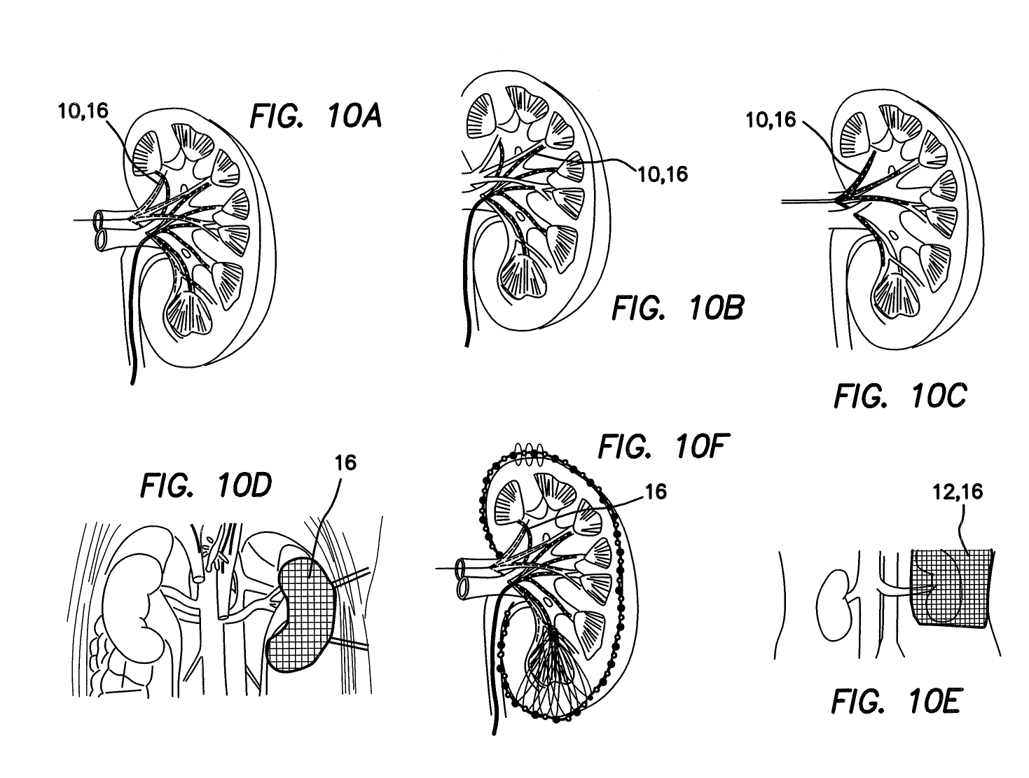

A method for drug, gene, siRNA, shRNA, peptide, protein, antibody or any other biomedical therapeutic molecules and reagents delivery in kidney in vivo is similar as shown in FIGS. 10.Ia, 10.Ib, 10.Ic, 10.IIa, 10.IIb and 10.III. In the in vivo embodiment, the negative electrode array mesh 12 is placed on the surface of the kidney endoscopically or during the abdominal surgery or an alternating array 16 is so disposed. The positive electrode array mesh 10 is placed into the renal pelvis through the urinary tract. The drug, gene, siRNA, shRNA, peptide, protein, antibody or any other biomedical therapeutic molecules and reagents are infused into the renal vessels, renal artery or vein. During and after the drug infusion, the electric field is applied on the targeted whole kidney. The electric field network extends through the kidney tissue. The positive and negative electrode positions are exchanged according the physical and chemical characteristic of drug, gene, siRNA, shRNA, peptide, protein, antibody or any other biomedical therapeutic molecules and reagents for obtaining the optimal delivery efficiency.

Alternatively, the positive electrode(s) 10 are also placed into the vessels of the kidney, such as renal artery or vein percutaneously as shown in FIG. 10.Ic or an alternating bipolar array 16 or negative array 12 is place on the skin surface in the proximate area as shown in FIG. 10.IIb. The drug, gene, siRNA, shRNA, peptide, protein, antibody or any other biomedical therapeutic molecules and reagents are also infused retrograde into the renal duct, and using a balloon to block the urinary out flow. Thus, the drug, gene, siRNA, shRNA, peptide, protein, antibody or any other biomedical therapeutic molecules and reagents remain in the kidney tissue for a relatively longer time.

Another alternative is to place the negative electrode array mesh 12 on the abdominal surface, while the positive electrode array mesh 10 is place into the renal pelvis as shown in FIG. 10.Ia. The drug, gene, siRNA, shRNA, peptide, protein, antibody or any other biomedical therapeutic molecules and reagents are infused retrograde into the renal vessels, such artery or vein percutaneously, or into urinary duct.

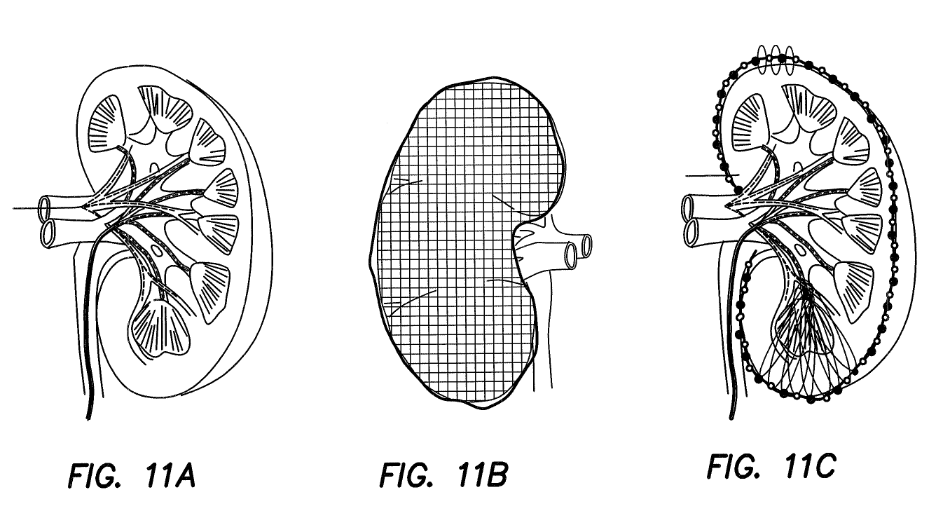

For ex vivo drug, gene, siRNA, shRNA, peptide, protein, antibody or any other biomedical therapeutic molecules and reagents delivery to the kidney, when the kidney is in the outside of the body as shown in FIGS. 11.I, 11.II, and 11.III, the negative electrode array mesh 12 is placed on the surface of the kidney directly as seen in FIG. 11.II. The positive electrode array mesh 10 is placed into the renal pelvis. The drug, gene, siRNA, shRNA, peptide, protein, antibody or any other biomedical therapeutic molecules and reagents are infused into the renal vessels, such as renal artery or vein, or infused retrograde into urinary tract. During and after the drug infusion, the electric field is applied to the targeted whole kidney as seen in FIG. 11.III. The electric field network extends across the kidney tissue.

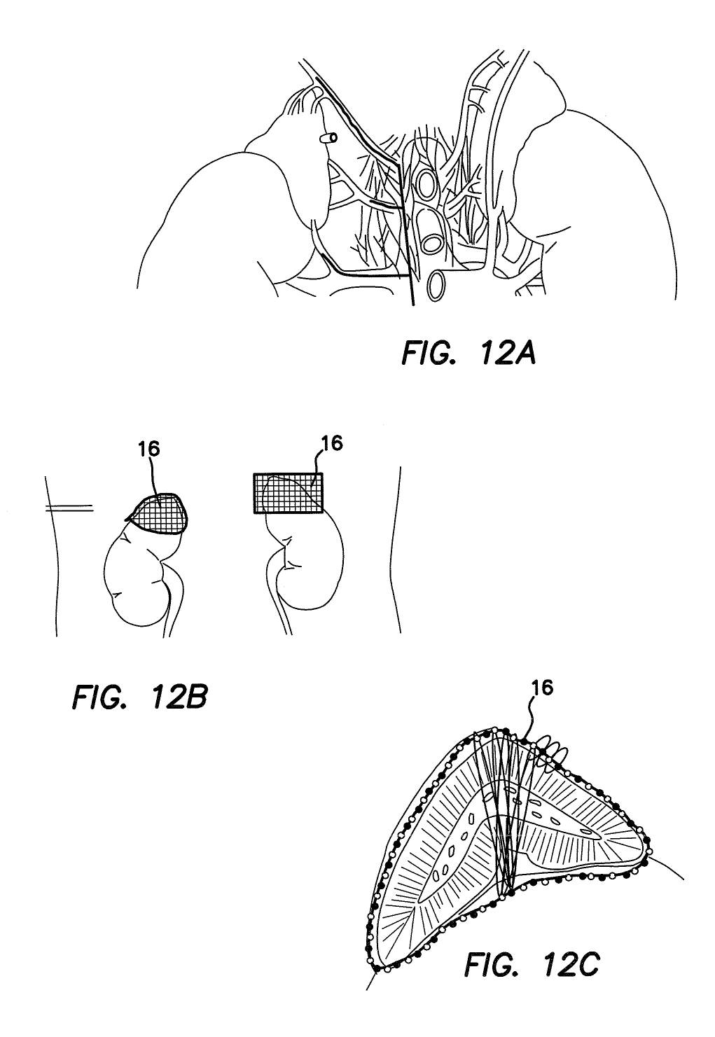

Still further the method for drug, gene, siRNA, shRNA, peptide, protein, antibody or any other biomedical therapeutic molecules and reagents delivery in the adrenal tissue as shown in FIGS. 12.I, 12.IIa, 12.IIb and 12.III contemplates placing the negative electrode array mesh 12 on the surface of the adrenal endoscopically or during the abdominal surgery. The positive electrode(s) 10 are placed in suprenal vessels, such as artery or vein, percutaneously or directly. Alternatively, a bipolar array 16 is applied on the adrenal tissue or on the proximate body surface as seen in FIGS. 12.IIa, 12.IIb and 12.III. The drug, gene, siRNA, shRNA, peptide, protein, antibody or any other biomedical therapeutic molecules and reagents are infused into the suprenal vessels. During and after the drug infusion, the electric field is applied on the whole or targeted part of the adrenal tissue. The electric field network extends through the adrenal tissue.

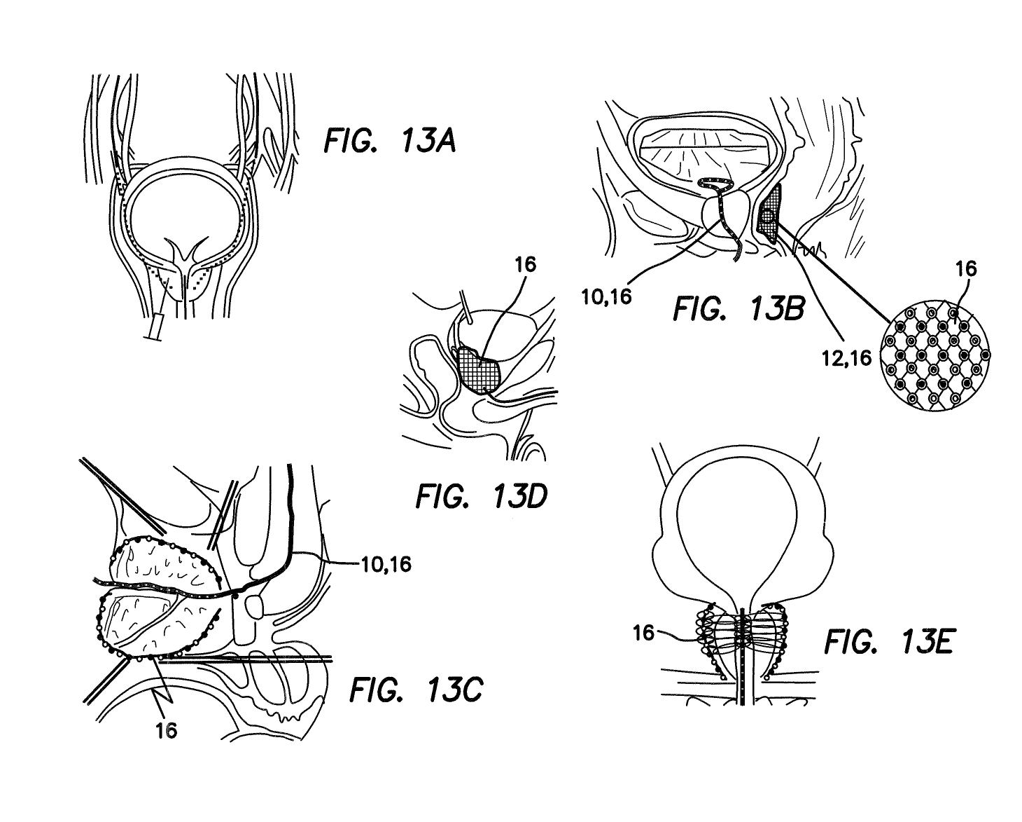

In the embodiment for delivery of drug, gene, siRNA, shRNA, peptide, protein, antibody or any other biomedical therapeutic molecules and reagents to the prostate as shown in FIGS. 13.Ia, 13.Ib, 13.IIa, 13.IIb, 13.III, 13.IIIa, and 13.IIIb, the negative electrode array mesh 12 is placed on the surface of the prostate endoscopically or during the abdominal surgery. The positive electrode 10 is placed into prostatic urethra through urinary tract. Alternatively in the embodiments shown in FIGS. 13.Ia, 13.Ib, 13.IIa, 13.IIb, 13.III, 13.IIIa, and 13.IIIb a bipolar array 16 is employed. The drug, gene, siRNA, shRNA, peptide, protein, antibody or any other biomedical therapeutic molecules and reagents are infused retrograde into the prostate through prostatic urethra or through prostatic vessels percutaneously. During and after the drug infusion, the electric field is applied on the whole or targeted part of the prostate. The electric field network extends through the prostate tissue.

Alternatively, the negative electrode array mesh 12 is placed on the abdominal surface, while the positive electrode array mesh 10 is placed into the prostatic urethra as seen in FIG. 13.IIa. The drug, gene, siRNA, shRNA, peptide, protein, antibody or any other biomedical therapeutic molecules and reagents are infused retrograde into the prostate through prostatic urethra or through prostatic vessels percutaneously.

Another alternative is to place the negative electrode array mesh 12 on to the surface of the prostate through rectal puncture.

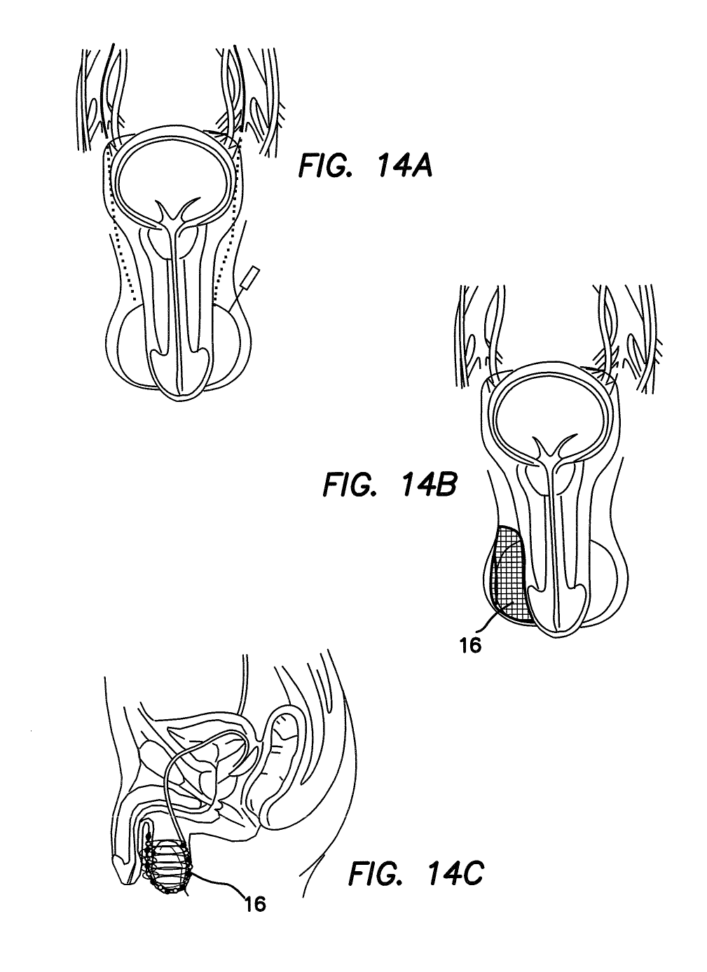

The embodiment for drug, gene, siRNA, shRNA, peptide, protein, antibody or any other biomedical therapeutic molecules and reagents delivery to the testicle as shown in FIGS. 14.Ia, 14.Ib, 14.IIa, and 14.III contemplates having half of the bipolar electrode array mesh 16 comprised of negative electrodes 12, and the other half with positive electrodes 10. Positive and negative electrodes are alternatively spatially arranged. The electrode array mesh 16 is placed on the surface of scrotum. The drug, gene, siRNA, shRNA, peptide, protein, antibody or any other biomedical therapeutic molecules and reagents are injected into the cavum serosum. After the drug infusion, the electric field is applied on the whole or targeted part of the testicle. The electric field network extends through the testicle tissue.

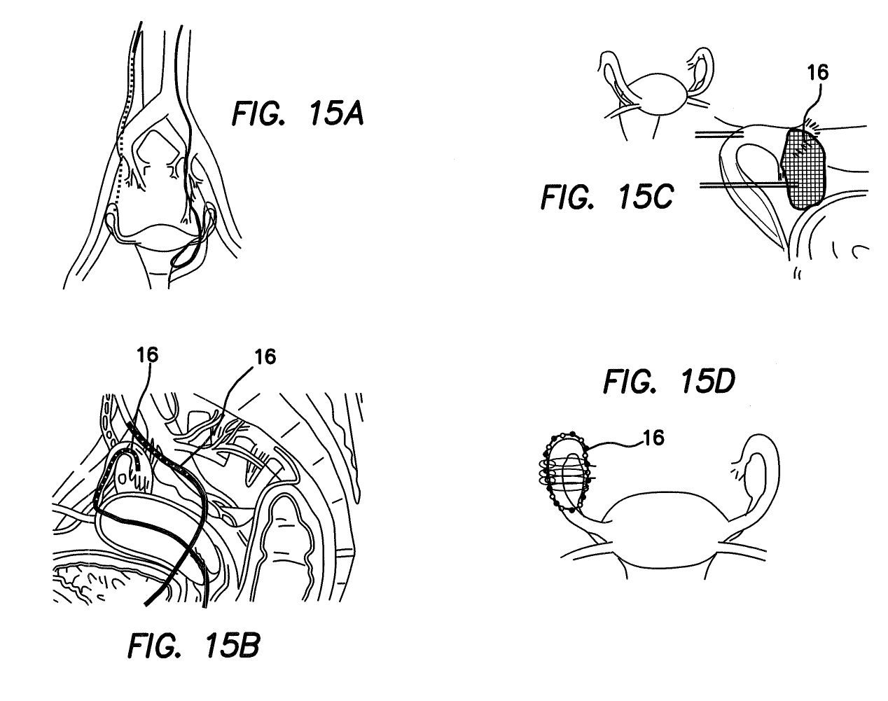

Similarly in the embodiment of the method for drug, gene, siRNA, shRNA, peptide, protein, antibody or any other biomedical therapeutic molecules and reagents delivery in ovary as shown in FIGS. 15.Ia, 15.Ib, 15.IIa, and 15.III, half of the bipolar electrode array mesh 16 is comprised of negative electrodes 12, and the other half with positive electrodes 10. Positive and negative electrodes are alternatively spatially arranged. The electrode array mesh 16 is placed on the surface of ovary endoscopically or during abdominal surgery. The drug, gene, siRNA, shRNA, peptide, protein, antibody or any other biomedical therapeutic molecules and reagents are released from a drug-retaining bag on or in association with the device or mesh 16. During and after the drug infusion, the electric field is applied on the whole or targeted part of the ovary. The electric field network extends through the ovarian tissue.

Alternatively, the electrode array mesh 16 is placed on the surface of the ovary noninvasively through vagina, uterus and Fallopian tube.

Another alternative is for the drug, gene, siRNA, shRNA, peptide, protein, antibody or any other biomedical therapeutic molecules and reagents to be infused through ovarian vessels.

The embodiment for drug, gene, siRNA, shRNA, peptide, protein, antibody or any other biomedical therapeutic molecules and reagents delivery into a chest, abdominal and pelvic tumor contemplates half of the bipolar electrode array mesh 16 comprised of negative electrodes 12, and half with positive electrodes 10. Positive and negative electrodes are alternatively spatially arranged. The electrode array mesh 16 is placed on the surface of a tumor endoscopically or during open-chest or abdominal surgery. The drug, gene, siRNA, shRNA, peptide, protein, antibody or any other biomedical therapeutic molecules and reagents are released from a drug-retaining bag coupled to mesh 16. During and after the drug infusion, the electric field is applied on the targeted tumor. The electric field network extends through the whole tumor.

Alternatively, alternative is the drug, gene, siRNA, shRNA, peptide, protein, antibody or any other biomedical therapeutic molecules and reagents are infused through vessels of the tumor.

Another alternative is to place the negative electrode array mesh 12 on the surface of the tumor and place a positive electrode 10 in the middle of the tumor. The drug, gene, siRNA, shRNA, peptide, protein, antibody or any other biomedical therapeutic molecules and reagents are injected into the tumor directly.

In the method and apparatus for gene, protein and drug delivery in:
1. the lung, the illustrated embodiment is used for the treatment of various lung diseases, such as various lung cancer or tumor, cystic fibrosis, emphysema, asthma, pulmonary hypertension, COPD, pulmonary embolism, pulmonary fibrosis, lung transplantation, lung A-V abnormalities etc.
2. the pleura, the method is used for the treatment of various pleural diseases, such as masothelioma, pleural fibrosis, various pleural malignancies.
3. the breast, it is used for the treatment of various breast malignancies, such as various breast cancer, various breast tumor.
4. the liver, it is used for the treatment of various liver diseases, such as various liver cancers, various kinds of liver tumors, various hepatitis, liver cirrhosis, liver abscess, liver transplantation, islet transplantation.
5. the spleen, it is used for the treatment of spleenomagaly, various splenic diseases.
6. the pancreas, it is used for the treatment of various pancreas malignancies, pancreas tumor, diabetes.
7. the kidney, it is used for the treatment of various kidney diseases, such as various kinds of kidney cancer and tumor, various kinds of autoimmune kidney diseases, nephritis, nephropathy, renal transplantation.
8. adrenal tissue, it is used for the treatment of various adrenal diseases, such as various adrenal cancers and tumors.
9. the prostate, it is used for the treatment of various prostate diseases, such as prostate cancer, prostate tumor, prostate hypertrophy.
10. the testicle, it is used for the treatment of various testicle diseases, such as testicle cancer, testicle tumor, testicle atrophy, infertility.
11. the ovary, it is used for the treatment of various ovarian diseases, such as ovarian cancer, ovarian tumor, ovarian neoplasms, ovarian cysts, premature ovarian failure, infertility.
12. tumors, it is used for the treatment of various chest, abdominal and pelvic tumors.

The illustrated embodiments of the invention open a new era for the ex vivo or in vivo delivering any therapeutic gene, siRNA, shRNA, shRNA protein or drugs in lung, pleura, breast, liver, spleen, pancreas, kidney, adrenal, prostate, testicle, ovary and tumors in chest, abdominal and pelvic cavity for prevention and treatment of large animal and human disease in vivo and ex vivo. There is no existing methodology which is as efficient and safe for the intracellular drug delivery localized in the targeted organ and that is applicable for human use.

The illustrated embodiments have four major advantages: 1) low voltage is used thereby reducing the cell damage; 2) more pulses and longer times are applied for increasing the gene and drug delivery efficiency; 3) more even distribution and homogenous strength of electrical field is applied to the tissue by using electric field network; and 4) better electrodes-tissue contact is achieved which that reduces the applied energy and significantly reduces tissue damage.

The illustrated embodiments of the invention is used for the treatment of various diseases in various solid organs, such as cancer. Currently, the successful treatment of these diseases always be limited by the inefficient local drug delivery and systemic drug use induced adverse effects. There is no better strategy in existence to overcome this problem. The illustrated method is safe, cost-effective and easy to develop.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

I claim:

1. A method comprising:
electropermeabilizing a solid organ or tissue mass of a large animal or human by means of application of a low strength electric field network (LSEN); and delivering a drug, gene, siRNA, shRNA, peptide, protein, antibody or a biomedical therapeutic reagent into the solid organ or tissue mass before, during or after application of LSEN.

2. The method of claim 1 where electropermeabilizing and delivering are performed ex vivo.

3. The method of claim 1 where electropermeabilizing and delivering are performed in vivo.

4. The method of claim 1 where electropermeabilizing the solid organ or tissue mass comprises electropermeabilizing a lung, pleura, breast, liver, spleen, pancreas, kidney, adrenal tissue, prostate, testicle, ovary or tumor.

5. The method of claim 4 where electropermeabilizing the lung comprises:
disposing at least one LSEN positive electrode into a vessel in the lung by percutaneously inserting or directly placing the LSEN positive electrode into pulmonary artery or its one or more of its branches during surgery;
disposing a LSEN negative electrode array outside of the lung through thoracoscopy or directly placing the LSEN negative electrode array on the outside of the lung during open-chest surgery;
infusing a drug, gene, siRNA, shRNA, peptide, protein, antibody or biomedical therapeutic reagent into the lung through a pulmonary artery catheter; and
applying an LSEN electric field during the infusion.

6. The method of claim 5 further comprising localizing the electropermeabilizing n in vivo for delivery to one or two lobes of the lung, a part of the lobe of the lung, one lung, a part of the lung or two lungs by changing the size of the electrode arrays, and the position of the positive electrode(s) and the choice of the vessel for delivery.

7. The method of claim 4 where electropermeabilizing the lung comprises:
disposing at least one positive electrode into the respiratory tract, including a trachea, bronchus, bronchiole or alveolar duct instead of pulmonary artery or vein; and
delivering in vivo a gene, protein and drug through the respiratory tract instead of vessels, or injecting a gene, protein or drug directly into the pleura cavity.

8. The method of claim 4 where electropermeabilizing the lung comprises:
disposing a negative electrode array noninvasively on the outside of the chest, instead of into the chest cavity; and
disposing at least one positive electrode into a pulmonary vessel or respiratory tract.

9. The method of claim 4 where electropermeabilizing the lung comprises:
disposing ex vivo a negative electrode array directly on the outside surface of the lung; and
disposing at least one positive electrode either into a vessel of the lung, or respiratory tract of the lung.

10. The method of claim 4 where electropermeabilizing the pleura comprises:
disposing a negative electrode array noninvasively on the outside of the chest, instead of into the chest cavity; and
disposing at least one positive electrode into a pulmonary vessel or respiratory tract; and
where delivering a drug, gene, siRNA, shRNA, peptide, protein, antibody or a biomedical therapeutic reagent into the solid organ or tissue mass before, during or after electropermeabilization comprises directly injecting the drug, gene, siRNA, shRNA, peptide, protein, antibody or any other biomedical therapeutic molecules and reagents into the pleura cavity, applying LSEN on the pleura during and after injection.

11. The method of claim 4 where delivering a drug, gene, siRNA, shRNA, peptide, protein, antibody or a biomedical therapeutic reagent into the solid organ or tissue mass before, during or after electropermeabilization comprises directly injecting a drug, gene, siRNA, shRNA, peptide, protein, antibody or a biomedical therapeutic molecule and reagent, and
where electropermeabilizing the pleura comprises disposing a plurality of positive and negative electrodes spatially arranged so that an electrode array mesh is disposed into the chest cavity thoraciscopically, and applying LSEN during and after the drug, gene, siRNA, shRNA, peptide, protein, antibody or a biomedical therapeutic molecule and reagent is directly injected into the pleura cavity.

12. The method of claim 4 where electropermeabilizing the breast comprises:
disposing a negative electrode array on the whole or partial breast in contact with skin;
disposing at least one positive electrode into a proximate vessel; and
applying LSEN during the infusion or after the injection in the whole or part of the breast which was targeted, and
where delivering a drug, gene, siRNA, shRNA, peptide, protein, antibody or a biomedical therapeutic reagent into the solid organ or tissue mass before, during or after electropermeabilization comprises infusing a drug, gene, siRNA, shRNA, peptide, protein, antibody or biomedical therapeutic molecule and reagent into proximate vessels or directly injecting the same into a targeted area of breast.

13. The method of claim 4 where electropermeabilizing the breast comprises disposing at least one positive electrode into a milk duct, and
where delivering a drug, gene, siRNA, shRNA, peptide, protein, antibody or a biomedical therapeutic reagent into the solid organ or tissue mass before, during or after electropermeabilization comprises infusing a drug, gene, siRNA, shRNA, peptide, protein, antibody or biomedical therapeutic molecule and reagent into the same or a different milk duct.

14. The method of claim 4 where electroporating the liver comprises:
disposing a negative electrode array on the surface of the liver endoscopically or surgically during the abdominal surgery;
disposing at least one positive electrode in a vessel of the liver, such as a hepatic artery or vein percutaneously; and
applying LSEN during and after drug infusion to the targeted whole or part of the liver, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or different hepatic vessel.

15. The method of claim 4 where electropermeabilizing the liver comprises:
disposing a negative electrode array mesh on the skin or body surface proximate to the liver;
disposing at least one positive electrode in a vessel of the liver, such as a hepatic artery or vein percutaneously; and
applying LSEN during and after the drug infusion to the targeted whole or part of the liver, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or a different hepatic vessel.

16. The method of claim 15 where disposing the at least one positive electrode in the vessel of the liver comprises disposing the at least one positive electrode into a portal vein, and where infusing the drug, gene, siRNA, shRNA, peptide, protein, antibody or biomedical therapeutic molecule and reagent into a hepatic vessel comprises infusing the drug, gene, siRNA, shRNA, peptide, protein, antibody or biomedical therapeutic molecule and reagent into the same or different portal vein.

17. The method of claim 4 where electropermeabilizing the liver comprises:
disposing ex vivo a negative electrode array on the surface of the liver directly;
disposing at least one positive electrode in a vessel of the liver, such as a hepatic artery, vein or portal vein; and
applying LSEN during and after the drug infusion to the whole or a targeted part of the liver, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or a different hepatic vessel.

18. The method of claim 4 where electropermeabilizing the spleen comprises:
disposing a negative electrode array on a surface of the spleen endoscopically or during the abdominal surgery;
disposing at least one positive electrode in a splenic vessel percutaneously or directly; and
applying LSEN during and after the drug infusion to the whole or targeted part of the spleen, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or a different splenic vessel.

19. The method of claim 4 where electropermeabilizing the pancreas comprises:
disposing a negative electrode array on the surface of the pancreas endoscopically or during the abdominal surgery;
disposing at least one positive electrode is disposed in a vessel of the pancreas percutaneously; and
applying LSEN during and after the drug infusion to the whole targeted pancreas, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or a different pancreatic vessel.

20. The method of claim 19 where electropermeabilizing the pancreas comprises disposing the at least one positive electrode through a pancreatic duct during abdominal surgery or through the intestines and where infusing the drug, gene, siRNA, shRNA, peptide, protein, antibody or biomedical therapeutic molecule and reagent into the same or a different pancreatic duct.

21. The method of claim 4 where electropermeabilizing the pancreas comprises:
disposing ex vivo a negative electrode array on a surface of the pancreas directly;
disposing at least one positive electrode in a pancreatic vessel or duct; and
applying LSEN during and after the drug infusion to the targeted whole or part of the pancreas, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the pancreatic vessels or duct.

22. The method of claim 4 where electropermeabilizing the kidney comprises:
disposing in vivo a negative electrode array mesh on a surface of the kidney endoscopically or during the abdominal surgery;
disposing a positive electrode array mesh into the renal pelvis through the urinary tract; and
applying LSEN during and after the drug infusion to the targeted whole kidney, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into a renal vessel, renal artery or vein.

23. The method of claim 4 where electropermeabilizing the kidney comprises:
disposing in vivo a negative electrode array mesh on a surface of the kidney endoscopically or during the abdominal surgery;
disposing at least one positive electrode into a vessel of the kidney, such as a renal artery or vein percutaneously; and
applying LSEN during and after the drug infusion to the targeted whole kidney
where delivering a drug, gene, siRNA, shRNA, peptide, protein, antibody or a biomedical therapeutic reagent into the solid organ or tissue mass before, during or after electropermeabilization comprises:
using a balloon to block the urinary out flow; and
retrograde infusing the drug, gene, siRNA, shRNA, peptide, protein, antibody or biomedical therapeutic molecule and reagent into a renal duct.

24. The method of claim 4 where electropermeabilizing the kidney comprises:
disposing a positive electrode array mesh into the renal pelvis;
disposing a negative electrode array mesh on an abdominal surface, while the positive electrode array mesh is disposed into the renal pelvis; and
applying LSEN during and after the drug infusion to the targeted whole kidney, and
where delivering a drug, gene, siRNA, shRNA, peptide, protein, antibody or a biomedical therapeutic reagent into the solid organ or tissue mass before, during or after electropermeabilization comprises retrograde infusing the drug, gene, siRNA, shRNA, peptide, protein, antibody or biomedical therapeutic molecule and reagent into a renal vessel, such an artery or vein percutaneously, or into a urinary duct.

25. The method of claim 4 where electropermeabilizing the kidney comprises:
disposing ex vivo a negative electrode array mesh on a surface of the kidney directly;
disposing the positive electrode array mesh into a renal pelvis; and
applying LSEN during and after the drug infusion to the targeted whole kidney, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into a renal vessel, such as a renal artery or vein, or retrograde infusing into the urinary tract.

26. The method of claim 4 where electropermeabilizing the adrenal tissue comprises:
disposing a negative electrode array mesh on a surface of the adrenal endoscopically or during the abdominal surgery;
disposing at least one positive electrode in a suprenal vessel, such as an artery or vein, percutaneously or directly; and
applying LSEN during and after the infusion to the whole or targeted part of the adrenal tissue, where the electric field network extends through the adrenal tissue, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent into the same or a different suprenal vessel.

27. The method of claim 4 where electropermeabilizing the prostate comprises:
disposing a negative electrode array mesh on a surface of the prostate endoscopically or during the abdominal surgery;
disposing at least one positive electrode into the prostatic urethra through urinary tract; and
applying LSEN during and after the infusion to the whole or targeted part of the prostate, and
where delivering a drug, gene, siRNA, shRNA, peptide, protein, antibody or a biomedical therapeutic reagent into the solid organ or tissue mass before, during or after electropermeabilization comprises retrograde infusing the drug, gene, siRNA, shRNA, peptide, protein, antibody or biomedical therapeutic molecule and reagent into the prostate through the prostatic urethra or through a prostatic vessel percutaneously.

28. The method of claim 4 where electropermeabilizing the prostate comprises:
disposing a positive electrode array mesh into the prostatic urethra;
disposing a negative electrode array mesh on an abdominal surface, while the positive electrode array mesh is disposed into the prostatic urethra; and
applying LSEN during and after the infusion to the whole or targeted part of the prostate, and
where delivering a drug, gene, siRNA, shRNA, peptide, protein, antibody or a biomedical therapeutic reagent into the solid organ or tissue mass before, during or after electropermeabilization comprises retrograde infusing the drug, gene, siRNA, shRNA, peptide, protein, antibody or biomedical therapeutic molecule and reagent into the prostate through prostatic urethra or through a prostatic vessel percutaneously.

29. The method of claim 4 where electropermeabilizing the prostate comprises:
disposing a positive electrode array mesh into the prostatic urethra;
disposing a negative electrode array mesh on a surface of the prostate through a rectal puncture, while the positive electrode array mesh is disposed into the prostatic urethra; and
applying LSEN during and after the infusion to the whole or targeted part of the prostate, and
where delivering a drug, gene, siRNA, shRNA, peptide, protein, antibody or a biomedical therapeutic reagent into the solid organ or tissue mass before, during or after electropermeabilization comprises retrograde infusing the drug, gene, siRNA, shRNA, peptide, protein, antibody or biomedical therapeutic molecule and reagent into the prostate through prostatic urethra or through a prostatic vessel percutaneously.

30. The method of claim 4 where electropermeabilizing the testicle comprises:
disposing an electrode mesh having half of the electrode array mesh comprised of negative electrodes, and the other half with positive electrodes alternatively spatially arranged on a surface of scrotum; and
applying LSEN after the infusion to the whole or targeted part of the testicle, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent or injecting the same into the cavum serosum.

31. The method of claim 4 where electropermeabilizing the ovary comprises:
disposing an electrode mesh having half of the electrode array mesh comprised of negative electrodes, and the other half with positive electrodes alternatively spatially arranged on the surface of the ovary endoscopically or during abdominal surgery; and
applying LSEN to the whole or targeted part of the ovary during and after the release, and
where delivering a drug, gene, siRNA, shRNA, peptide, protein, antibody or a biomedical therapeutic reagent into the solid organ or tissue mass before, during or after electropermeabilization comprises releasing the drug, gene, siRNA, shRNA, peptide, protein, antibody or biomedical therapeutic molecule and reagent from a drug-retaining bag on the mesh.

32. The method of claim 4 where electropermeabilizing the ovary comprises:
disposing an electrode mesh having half of the electrode array mesh comprised of negative electrodes, and the other half with positive electrodes alternatively spatially arranged on a surface of the ovary noninvasively through vagina, uterus and Fallopian tube; and
applying LSEN to the whole or targeted part of the ovary during and after the release, and
where delivering a drug, gene, siRNA, shRNA, peptide, protein, antibody or a biomedical therapeutic reagent into the solid organ or tissue mass before, during or after electropermeabilization comprises releasing the drug, gene, siRNA, shRNA, peptide, protein, antibody or biomedical therapeutic molecule and reagent from a drug-retaining bag on the mesh.

33. The method of claim 4 where electropermeabilizing the ovary comprises:
disposing an electrode mesh having half of the electrode array mesh comprised of negative electrodes, and the other half with positive electrodes alternatively spatially arranged on the surface of the ovary; and
applying LSEN to the whole or targeted part of the ovary during and after the release, and
where delivering a drug, gene, siRNA, shRNA, peptide, protein, antibody or a biomedical therapeutic reagent into the solid organ or tissue mass before, during or after electropermeabilization comprises injecting the drug, gene, siRNA, shRNA, peptide, protein, antibody or biomedical therapeutic molecule and reagent through an ovarian vessel.

34. The method of claim 4 where electropermeabilizing a chest, abdominal and pelvic tumor comprises:
disposing an electrode mesh having half of the electrode array mesh comprised of negative electrodes, and the other half with positive electrodes alternatively spatially arranged on the surface of the tumor endoscopically or during open-chest or abdominal surgery; and
applying LSEN to the targeted tumor during and after the release, and
where delivering a drug, gene, siRNA, shRNA, peptide, protein, antibody or a biomedical therapeutic reagent into the solid organ or tissue mass before, during or after electropermeabilization comprises releasing the drug, gene, siRNA, shRNA, peptide, protein, antibody or biomedical therapeutic molecule and reagent from a drug-retaining bag coupled to the mesh.

35. The method of claim 4 where electropermeabilizing a chest, abdominal and pelvic tumor comprises:
disposing an electrode mesh having half of the electrode array mesh comprised of negative electrodes, and the other half with positive electrodes alternatively spatially arranged on a surface of the tumor endoscopically or during open-chest or abdominal surgery; and
applying LSEN to the targeted tumor during and after the release, and
where delivering a drug, gene, siRNA, shRNA, peptide, protein, antibody or a biomedical therapeutic reagent into the solid organ or tissue mass before, during or after electropermeabilization comprises infusing the drug, gene, siRNA, shRNA, peptide, protein, antibody or biomedical therapeutic molecule and reagent through a vessel of the tumor.

36. The method of claim 4 where electropermeabilizing a chest, abdominal and pelvic tumor comprises:
disposing a negative electrode array mesh on a surface of the tumor;
disposing a positive electrode in the tumor; and
applying LSEN to the targeted tumor during and after the release, and
where delivering a drug, gene, siRNA, shRNA, peptide, protein, antibody or a biomedical therapeutic reagent into the solid organ or tissue mass before, during or after electropermeabilization comprises directly injecting the drug, gene, siRNA, shRNA, peptide, protein, antibody or biomedical therapeutic molecule and reagent into the tumor.

37. The methods of claim 1 where electropermeabilizing a solid organ or tissue mass of a large animal or human by means of application of a low strength electric field network (LSEN) comprises using positive and negative electrodes to apply the LSEN and further comprising exchanging the positions of the positive and negative electrodes according the physical and chemical characteristic of the drug, gene, siRNA, shRNA, peptide, protein, antibody or biomedical therapeutic molecule and reagent for obtaining the optimal delivery efficiency.

38. An apparatus for delivering a drug, gene, siRNA, shRNA, peptide, protein, antibody or a biomedical therapeutic reagent into the solid organ or tissue mass comprising:
means for electropermeabilizing a solid organ or tissue mass of a large animal or human by means of application of a low strength electric field network (LSEN); and
means for delivering the drug, gene, siRNA, shRNA, peptide, protein, antibody or a biomedical therapeutic reagent into the solid organ or tissue mass before, during or after electropermeabilization.

39. An apparatus for use in a method comprising:
electropermeabilizing a solid organ or tissue mass of a large animal or human by means of application of a low strength electric field network (LSEN); and
delivering the drug, gene, siRNA, shRNA, peptide, protein, antibody or a biomedical therapeutic reagent into the solid organ or tissue mass before, during or after electropermeabilization.

40. An apparatus for use in a method comprising:
an electrode mesh and/or electrode in combination for electropermeabilizing a solid organ or tissue mass of a large animal or human by means of application of a low strength electric field network (LSEN); and
an infuser or injection device for delivering the drug, gene, siRNA, shRNA, peptide, protein, antibody or a biomedical therapeutic reagent into the solid organ or tissue mass before, during or after electropermeabilization.
